(19) United States
(12) Patent Application Publication
(10) Pub. No.: US 2022/0205974 A1
(43) Pub. Date: Jun. 30, 2022

(54) GAS DETECTION SYSTEM

(71) Applicant: KYOCERA Corporation, Kyoto-shi, Kyoto (JP)

(72) Inventors: Shinichi ABE, Uji-shi, Kyoto (JP); Etsuro SHIMIZU, Uji-shi, Kyoto (JP); Daisuke UEYAMA, Nara-shi, Nara (JP)

(21) Appl. No.: 17/606,406

(22) PCT Filed: Apr. 20, 2020
(86) PCT No.: PCT/JP2020/017090

§ 371 Date: Oct. 25, 2021

(30) Foreign Application Priority Data
Apr. 26, 2019 (JP) 2019-086575

(51) Int. Cl. G01N 33/497; E03D 9/00; G01N 1/22; G01N 27/416; G01N 33/00; F24F 7/003; E03D 9/04

(52) U.S. Cl. CPC G01N 33/497; E03D 9/005; G01N 1/2273; G01N 1/2226; G01N 27/4162; G01N 33/0036; F24F 2110/65; G01N 33/0062; G01N 33/0073; G01N 1/2214; F24F 7/003; E03D 9/04; G01N 33/0011

(57) ABSTRACT

A gas detection system includes a first sensor unit that outputs a voltage corresponding to a concentration of a specific gas, a storage tank capable of storing a sample gas or a purge gas to be supplied to the first sensor unit, and a control unit. The control unit detects a type and a concentration of a gas contained in the sample gas on the basis of a detection result of the first sensor unit. The control unit sets a collection period during which a gas in a predetermined space is collected into the storage tank as the sample gas or the purge gas and a supply period during which the sample gas or the purge gas is supplied to the first sensor unit such that the collection period and the supply period fall in different time slots.

### CROSS REFERENCE TO RELATED APPLICATIONS

The present application claims priority to Japanese Patent Application No. 2019-086575 filed in Japan on Apr. 26, 2019, the entire disclosure of which is incorporated herein by reference.

### TECHNICAL FIELD

The present disclosure relates to a gas detection system.

### BACKGROUND ART

In the related art, there is known a system for detecting an odoriferous gas generated from feces discharged by a subject (for example, PTL 1).

### CITATION LIST

### Patent Literature

PTL 1: Japanese Unexamined Patent Application Publication No. 2016-142584

### SUMMARY OF INVENTION

A gas detection system according to an embodiment of the present disclosure includes:

a first sensor unit that outputs a voltage corresponding to a concentration of a specific gas;

a storage tank capable of storing a sample gas or a purge gas to be supplied to the first sensor unit; and

a control unit that detects a type and a concentration of a gas contained in the sample gas on the basis of a detection result of the first sensor unit, wherein

the control unit sets a collection period during which a gas in a predetermined space is collected into the storage tank as the sample gas or the purge gas and a supply period during which the sample gas or the purge gas is supplied to the first sensor unit such that the collection period and the supply period fall in different time slots.

A gas detection system according to an embodiment of the present disclosure includes:

a first sensor unit that outputs a voltage corresponding to a concentration of a specific gas;

a first storage tank capable of storing a purge gas to be supplied to the first sensor unit; and

a control unit that collects a gas in a predetermined space into the first storage tank as the purge gas when a ventilation fan installed in the predetermined space is in a driven state, wherein

the ventilation fan is capable of exchanging between the gas in the predetermined space and a gas outside the predetermined space.

A gas detection system according to an embodiment of the present disclosure includes:

a first sensor unit that outputs a voltage corresponding to a concentration of a specific gas;

a first storage tank capable of storing a purge gas to be supplied to the first sensor unit; and

a control unit that detects a type and a concentration of a gas contained in the sample gas on the basis of a detection result of the first sensor unit, wherein

the control unit

executes first refresh processing on the first sensor unit by using a gas remaining in the first storage tank or by using a gas in a predetermined space, and

executes second refresh processing on the first sensor unit by using the purge gas stored in the first storage tank.

### BRIEF DESCRIPTION OF DRAWINGS

FIG. 1 is an external view of a gas detection system according to a first embodiment of the present disclosure.

FIG. 2 is a schematic diagram of the gas detection system illustrated in FIG. 1.

FIG. 3 is a functional block diagram of the gas detection system illustrated in FIG. 1.

FIG. 4 is a flowchart illustrating the operation of the gas detection system illustrated in FIG. 1.

FIG. 5 is a flowchart illustrating an example operation of the gas detection system according to the first embodiment of the present disclosure during collection of a purge gas.

FIG. 6 is a flowchart illustrating another example operation of the gas detection system according to the first embodiment of the present disclosure during collection of a purge gas.

FIG. 7 is a flowchart illustrating still another example operation of the gas detection system according to the first embodiment of the present disclosure during collection of a purge gas.

FIG. 8 is a flowchart illustrating the operation of a gas detection system according to a second embodiment of the present disclosure during refresh.

FIG. 9 is a functional block diagram of a gas detection system according to a modification of the present disclosure.

### DESCRIPTION OF EMBODIMENTS

Conventional systems are susceptible to improvement.

The present disclosure relates to providing an improved gas detection system.

According to an embodiment of the present disclosure, an improved gas detection system can be provided.

Embodiments according to the present disclosure will be described hereinafter with reference to the drawings schematically illustrating the embodiments.

### First Embodiment

[Example Configuration of Gas Detection System]

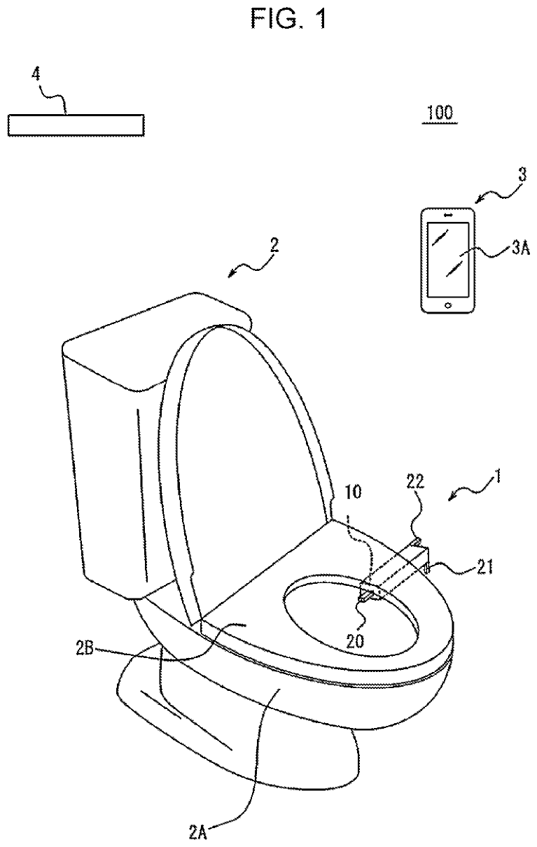

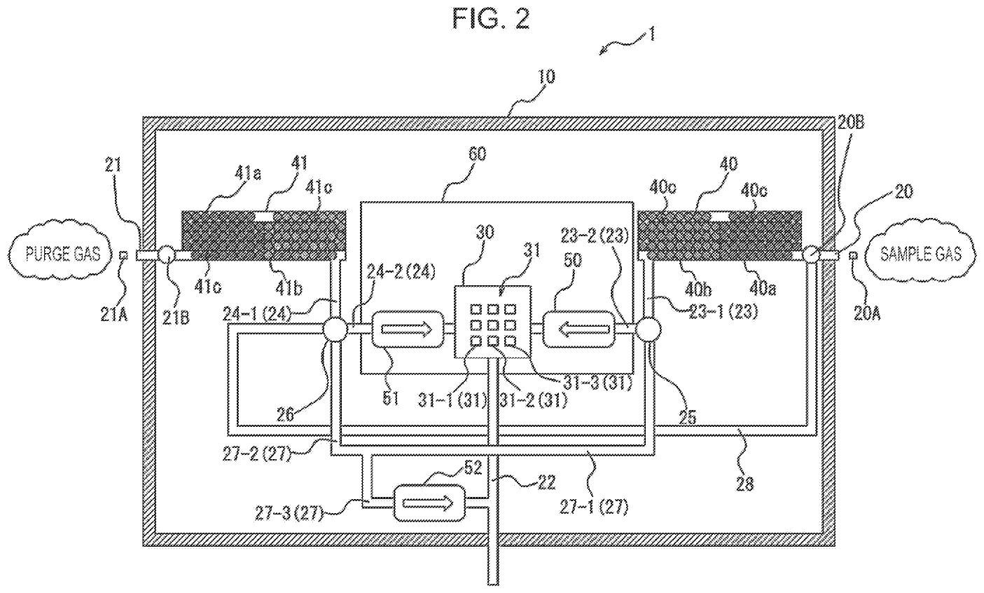

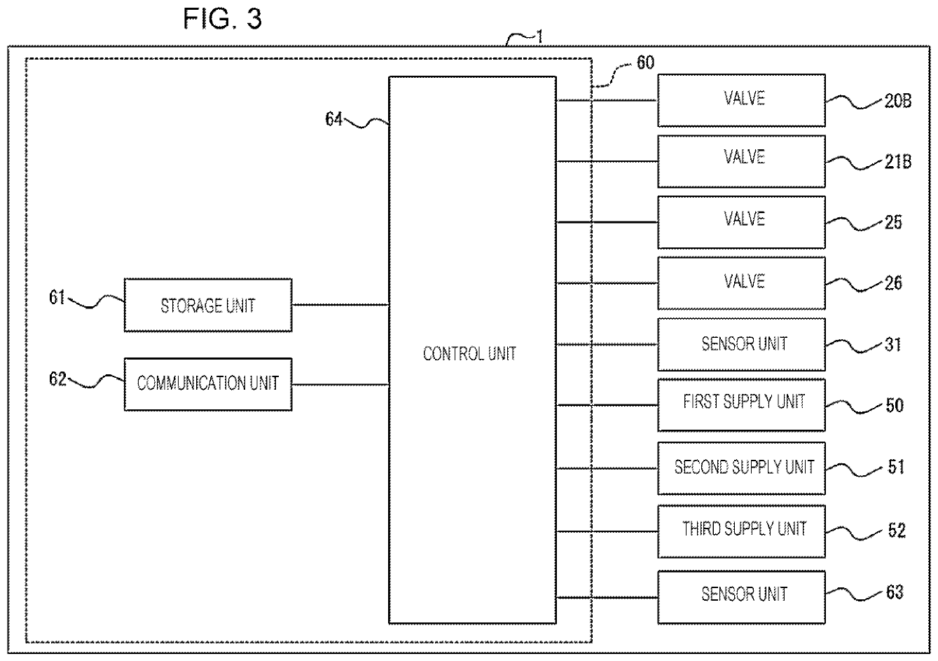

FIG. 1 is an external view of a gas detection system 1 according to a first embodiment of the present disclosure. FIG. 2 is a schematic diagram of the gas detection system 1 illustrated in FIG. 1. FIG. 2 illustrates a state in which a portion of a housing 10 included in the gas detection system 1 is removed. FIG. 3 is a functional block diagram of the gas detection system 1 illustrated in FIG. 1.

As illustrated in FIG. 1, the gas detection system 1 is installed in a toilet room 100 (predetermined space). In this embodiment, the predetermined space in which the gas detection system 1 is arranged is the toilet room 100 as illustrated in FIG. 1. However, the predetermined space in the present disclosure is not limited to the toilet room 100. The predetermined space may be any space as long as a gas to be detected by a gas detection system according to the present disclosure can be generated in the space. The gas detection system 1 is also referred to as a “gas detection device”.

As illustrated in FIG. 1, the gas detection system 1 is installed in a toilet 2. The toilet 2 may be, but is not limited to, a flush toilet. The gas detection system 1 may be installed in any portion of the toilet 2. In one example, as illustrated in FIG. 1, the gas detection system 1 may be arranged from between a toilet bowl 2A and a toilet seat 2B to the outside of the toilet 2. A portion of the gas detection system 1 may be embedded inside the toilet seat 2B. A subject can discharge feces into the toilet bowl 2A of the toilet 2. The gas detection system 1 can acquire a gas generated from the feces discharged into the toilet bowl 2A as a sample gas. The gas detection system 1 can detect the type of a gas contained in the sample gas, the concentration of the gas, and so on. The gas detection system 1 can transmit the detection results and so on to an electronic device 3.

The uses of the gas detection system 1 are not limited to the use described above. For example, the gas detection system 1 may be installed in a refrigerator as a predetermined space. In this case, the gas detection system 1 can acquire a gas generated from food as a sample gas. For example, the gas detection system 1 may be installed in a factory or a laboratory as a predetermined space. In this case, the gas detection system 1 can acquire a gas generated from a chemical or the like as a sample gas.

The toilet 2 can be installed in the toilet room 100 in a house, a hospital, or the like. The toilet 2 can be used by the subject. The toilet 2 includes the toilet bowl 2A and the toilet seat 2B. The subject can discharge feces into the toilet bowl 2A.

The electronic device 3 is, for example, a smartphone used by the subject. However, the electronic device 3 is not limited to the smartphone and may be any electronic device. When brought into the toilet room 100 by the subject, as illustrated in FIG. 1, the electronic device 3 can be present in the toilet room 100. However, for example, when the subject does not bring the electronic device 3 into the toilet room 100, the electronic device 3 may be present outside the toilet room 100. The electronic device 3 can receive the detection results from the gas detection system 1 via wireless communication or wired communication. The electronic device 3 can display the received detection results on a display unit 3A. The display unit 3A may include a display capable of displaying characters and the like, and a touch screen capable of detecting contact of a finger of the user (subject) or the like. The display may include a display device such as a liquid crystal display (LCD), an organic EL display (OELD: Organic Electro-Luminescence Display), or an inorganic EL display (IELD: Inorganic Electro-Luminescence Display). The detection method of the touch screen may be any method such as a capacitance method, a resistance film method, a surface acoustic wave method, an ultrasonic method, an infrared method, an electromagnetic induction method, or a load detection method.

A ventilation fan 4 may be installed on the ceiling of the toilet room 100. When the ventilation fan 4 is brought into a driven state, air in the toilet room 100 and air outside the toilet room 100 can be exchanged. The ventilation fan 4 may be capable of communicating with the gas detection system 1. The ventilation fan 4 may transmit a signal indicating the state of the ventilation fan 4 to the gas detection system 1. For example, when the ventilation fan 4 is in the driven state, the ventilation fan 4 may transmit a signal indicating that the ventilation fan 4 is in the driven state to the gas detection system 1. For example, when the ventilation fan 4 is not in a non-operating state, the ventilation fan 4 may transmit a signal indicating that the ventilation fan 4 is in the non-operating state to the gas detection system 1.

As illustrated in FIG. 2, the gas detection system 1 includes the housing 10, a suction hole 20, a suction hole 21, a discharge path 22, flow paths 23 and 24, a chamber 30, a supply unit 50, a supply unit 51, and a circuit board 60. The gas detection system 1 includes a storage tank capable of storing air in the toilet room 100 as illustrated in FIG. 1 (a gas in the predetermined space) as a sample gas or a purge gas. In this embodiment, the storage tank includes a storage tank 40 (second storage tank) capable of storing the sample gas, and a storage tank 41 (first storage tank) capable of storing the purge gas. However, the storage tank may include only one of the storage tank 40 and the storage tank 41. As illustrated in FIG. 2, the gas detection system 1 includes, in the chamber 30, a sensor unit 31 (first sensor unit). The flow path 23 includes a flow path 23-1 and a flow path 23-2. The flow path 24 includes a flow path 24-1 and a flow path 24-2. The gas detection system 1 may include a valve 20B and a valve 21B. The gas detection system 1 may include valves 25 and 26, a flow path 27, a flow path 28, and a supply unit 52. The flow path 27 includes a flow path 27-1, a flow path 27-2, and a flow path 27-3. As illustrated in FIG. 3, the gas detection system 1 includes, in the circuit board 60, a storage unit 61, a communication unit 62, and a control unit 64. The gas detection system 1 includes a sensor unit 63 (second sensor unit). The gas detection system 1 may further include a battery, a speaker, and the like.

The housing 10 houses various components of the gas detection system 1. The housing 10 may be made of any material. For example, the housing 10 may be made of a material such as metal or resin.

As illustrated in FIG. 1, the suction hole 20 can be exposed to the inside of the toilet bowl 2A. A portion of the suction hole 20 may be embedded in the toilet seat 2B. The suction hole 20 sucks in a gas generated from feces discharged into the toilet bowl 2A as a sample gas. The sample gas sucked in through the suction hole 20 is supplied to and stored in the storage tank 40 via the valve 20B as illustrated in FIG. 2. As illustrated in FIG. 1, one end of the suction hole 20 may be directed to the inside of the toilet bowl 2A. As illustrated in FIG. 2, the other end of the suction hole 20 may be connected to the storage tank 40. The suction hole 20 may be constituted by a tubular member such as a resin tube or a metal or glass pipe.

As illustrated in FIG. 2, the suction hole 20 may have, on the outside thereof, an air blower 20A. The air blower 20A may include a fan and a motor. The air blower 20A can drive the motor to rotate the fan under the control of the control unit 64. The air blower 20A rotates the fan to draw a gas generated from feces into around the suction hole 20. The air blower 20A draws a gas generated from feces into around the suction hole 20, the valve 25 connects the flow path 23-1 and the flow path 23-2 to each other, and the supply unit 50 is driven to allow the suction hole 20 to suck in the gas generated from the feces in the toilet bowl 2A.

The valve 20B is located among the suction hole 20, the storage tank 40, and the flow path 28. The valve 20B includes a connection port connected to the suction hole 20, a connection port connected to an inlet portion of the storage tank 40, and a connection port connected to the flow path 28. The valve 20B may be constituted by a valve such as an electromagnetically driven valve, a piezoelectrically driven valve, or a motor-driven valve.

The valve 20B switches the connection state among the suction hole 20, the storage tank 40, and the flow path 28 under the control of the control unit 64. For example, the valve 20B switches the connection state among them to a state in which the suction hole 20 and the storage tank 40 are connected to each other, a state in which the storage tank 40 and the flow path 28 are connected to each other, or a state in which the suction hole 20, the storage tank 40, and the flow path 28 are not connected to each other.

When the suction hole 20 sucks in the sample gas, the valve 20B connects the suction hole 20 and the storage tank 40 to each other under the control of the control unit 64. When the sample gas is stored in the storage tank 40, the valve 20B does not connect the suction hole 20, the storage tank 40, and the flow path 28 to each other under the control of the control unit 64. The valve 20B does not connect the storage tank 40 and the suction hole 20 to each other, which can reduce the probability that the sample gas in the storage tank 40 comes into contact with the outside air.

As illustrated in FIG. 1, the suction hole 21 can be exposed to the outside of the toilet bowl 2A. A portion of the suction hole 21 may be embedded in the toilet seat 2B. The suction hole 21 sucks in, for example, air (surrounding gas) in the toilet room 100, which is outside the toilet bowl 2A, as a purge gas. The purge gas sucked in through the suction hole 21 is supplied to and stored in the storage tank 41 via the valve 21B as illustrated in FIG. 2. As illustrated in FIG. 1, one end of the suction hole 21 may be directed to the outside of the toilet 2. As illustrated in FIG. 2, the other end of the suction hole 21 may be connected to the storage tank 41. The suction hole 21 may be constituted by a tubular member such as a resin tube or a metal or glass pipe.

As illustrated in FIG. 2, the suction hole 21 may have, on the outside thereof, an air blower 21A. The air blower 21A may include a fan and a motor. The air blower 21A can drive the motor to rotate the fan under the control of the control unit 64. The air blower 21A rotates the fan to draw the air in the toilet room 100 into around the suction hole 21. The air blower 21A draws the air in the toilet room 100 into around the suction hole 21, the valve 26 connects the flow path 24-1 and the flow path 24-2 to each other, and the supply unit 51 is driven to allow the suction hole 21 to suck in the air in the toilet room 100 as a purge gas.

The valve 21B is located between the suction hole 21 and the storage tank 41. The valve 21B includes a connection port connected to the suction hole 21, and a connection port connected to an inlet portion of the storage tank 41. The valve 21B may be constituted by a valve such as an electromagnetically driven valve, a piezoelectrically driven valve, or a motor-driven valve.

The valve 21B switches the connection state between the suction hole 21 and the storage tank 41 under the control of the control unit 64. For example, the valve 21B switches the connection state between them to a state in which the suction hole 21 and the storage tank 41 are connected to each other or a state in which the suction hole 21 and the storage tank 41 are not connected to each other.

When the suction hole 21 sucks in the purge gas, the valve 21B connects the suction hole 21 and the storage tank 41 to each other under the control of the control unit 64. When the purge gas is stored in the storage tank 41, the valve 21B does not connect the suction hole 21 and the storage tank 41 to each other under the control of the control unit 64. The valve 20B does not connect the storage tank 40 and the suction hole 20 to each other, which can reduce the probability that the purge gas in the storage tank 41 comes into contact with the outside air.

As illustrated in FIG. 1, a portion of the discharge path 22 can be exposed to the outside of the toilet bowl 2A. The discharge path 22 as illustrated in FIG. 2 discharges the exhaust from the chamber 30 to the outside. The exhaust can contain the sample gas and the purge gas, which have been subjected to detection processing. Further, the discharge path 22 can discharge the residual gas or the like in the storage tank 40 to the outside via the flow path 23-1, the valve 25, the flow paths 27-1 and 27-3, and the supply unit 52. Further, the discharge path 22 can discharge the residual gas or the like in the storage tank 41 to the outside via the flow path 24-1, the valve 26, the flow paths 27-2 and 27-3, and the supply unit 52. The discharge path 22 may be constituted by a tubular member such as a resin tube or a metal or glass pipe.

When the valve 25 connects the flow path 23-1 and the flow path 23-2 to each other, the flow path 23 as illustrated in FIG. 2 supplies the sample gas stored in the storage tank 40 to the chamber 30 via the supply unit 50. One end of the flow path 23-1 is connected to an outlet portion of the storage tank 40. The other end of the flow path 23-1 is connected to the valve 25. One end of the flow path 23-2 is connected to the valve 25. The other end of the flow path 23-2 is connected to the chamber 30. The flow path 23 may be constituted by a tubular member such as a resin tube or a metal or glass pipe.

When the valve 26 connects the flow path 24-1 and the flow path 24-2 to each other, the flow path 24 as illustrated in FIG. 2 supplies the purge gas stored in the storage tank 41 to the chamber 30 via the supply unit 51. One end of the flow path 24-1 is connected to an outlet portion of the storage tank 41. The other end of the flow path 24-1 is connected to the valve 26. One end of the flow path 24-2 is connected to the valve 26. The other end of the flow path 24-2 is connected to the chamber 30. The flow path 24 may be constituted by a tubular member such as a resin tube or a metal or glass pipe.

As illustrated in FIG. 2, the valve 25 is located among the flow path 23-1, the flow path 23-2, and the flow path 27-1. The valve 25 includes a connection port connected to the flow path 23-1, a connection port connected to the flow path 23-2, and a connection port connected to the flow path 27-1. The valve 25 may be constituted by a valve such as an electromagnetically driven valve, a piezoelectrically driven valve, or a motor-driven valve.

The valve 25 switches the connection state among the flow path 23-1, the flow path 23-2, and the flow path 27-1 under the control of the control unit 64. For example, the valve 25 switches the connection state among them to a state in which the flow path 23-1 and the flow path 23-2 are connected to each other or a state in which the flow path 23-1 and the flow path 27-1 are connected to each other.

As illustrated in FIG. 2, the valve 26 is located among the flow path 24-1, the flow path 24-2, and the flow path 27-2. The valve 26 includes a connection port connected to the flow path 24-1, a connection port connected to the flow path 24-2, and a connection port connected to the flow path 27-2. The valve 26 may be constituted by a valve such as an electromagnetically driven valve, a piezoelectrically driven valve, or a motor-driven valve.

The valve 26 switches the connection state among the flow path 24-1, the flow path 24-2, the flow path 27-2, and the flow path 28 under the control of the control unit 64. For example, the valve 26 switches the connection state among them to a state in which the flow path 24-1 and the flow path 24-2 are connected to each other, a state in which the flow path 24-1 and the flow path 27-2 are connected to each other, or a state in which the flow path 24-1 and the flow path 28 are connected to each other.

When the valve 25 connects the flow path 23-1 and the flow path 27-1 to each other, the flow path 27 as illustrated in FIG. 2 supplies the residual gas or the like in the storage tank 40 to the discharge path 22 via the supply unit 52. When the valve 26 connects the flow path 24-1 and the flow path 27-2 to each other, the flow path 27 supplies the residual gas or the like in the storage tank 41 to the discharge path 22 via the supply unit 52. One end of the flow path 27-1 is connected to the valve 25. The other end of the flow path 27-1 is connected to one end of the flow path 27-3. One end of the flow path 27-2 is connected to the valve 26. The other end of the flow path 27-2 is connected to the one end of the flow path 27-3. The one end of the flow path 27-3 is connected to the other end of the flow path 27-1 and the other end of the flow path 27-2. The other end of the flow path 27-3 is connected to the discharge path 22. The flow path 27 may be constituted by a tubular member such as a resin tube or a metal or glass pipe.

When the valve 26 connects the flow path 24-1 and the flow path 28 to each other and the valve 20B connects the flow path 28 and the storage tank 40 to each other, the flow path 28 as illustrated in FIG. 2 supplies the purge gas in the storage tank 41 to the storage tank 40. Since the purge gas is supplied to the storage tank 40 via the flow path 28, the sample gas in the storage tank 40 is pushed out into the flow path 23-1. One end of the flow path 28 is connected to the valve 20B. The other end of the flow path 28 is connected to the valve 26. The flow path 28 may be constituted by a tubular member such as a resin tube or a metal or glass pipe.

The chamber 30 as illustrated in FIG. 2 has, inside thereof, the sensor unit 31. The chamber 30 may include a plurality of sensor units 31. The chamber 30 may be divided into a plurality of chambers. The sensor units 31 may be disposed in the resulting plurality of chambers 30. The plurality of chambers 30 may be connected to each other. The chamber 30 is connected to the flow path 23-2. The sample gas is supplied to the chamber 30 from the flow path 23-2. The chamber 30 is also connected to the flow path 24-2. The purge gas is supplied to the chamber 30 from the flow path 24-2. The chamber 30 is further connected to the discharge path 22. The chamber 30 discharges the sample gas and the purge gas, which have been subjected to detection processing, from the discharge path 22.

The sensor unit 31 is arranged in the chamber 30. The sensor unit 31 outputs a voltage corresponding to the concentration of a specific gas to the control unit 64. The specific gas contains a specific gas to be detected and a specific gas not to be detected. When the sample gas is a gas generated from feces, examples of the specific gas to be detected include methane, hydrogen, carbon dioxide, methyl mercaptan, hydrogen sulfide, acetic acid, and trimethylamine. When the sample gas is a gas generated from feces, examples of the specific gas not to be detected include ammonia and water. Each of the plurality of sensor units 31 can output a voltage corresponding to the concentration of at least any one of these gases to the control unit 64.

As illustrated in FIG. 2, the storage tank 40 is connected to a connection port of the valve 20B. A portion of the storage tank 40 that is connected to the connection port of the valve 20B is also referred to as an “inlet portion”. The storage tank 40 is connected to the flow path 23-1. A portion of the storage tank 40 that is connected to the flow path 23-1 is also referred to as an “outlet portion”.

The storage tank 40 is capable of storing the sample gas. The sample gas stored in the storage tank 40 is supplied to the chamber 30 via the flow paths 23-1 and 23-2 and the supply unit 50. The residual gas or the like in the storage tank 40 can be discharged to the outside from the discharge path 22 via the flow path 23-1, the valve 25, the flow paths 27-1 and 27-3, and the supply unit 52.

An adsorbent 40a may be placed in the storage tank 40. Further, the sample gas may be concentrated in the storage tank 40. In this case, an adsorbent 40b may be placed in the storage tank 40. Each of the adsorbent 40a and the adsorbent 40b may contain any material corresponding to the use. Each of the adsorbent 40a and the adsorbent 40b may contain, for example, at least any one of activated carbon, silica gel, zeolite, and molecular sieve. The adsorbent 40a and the adsorbent 40b may be of a plurality of types or may contain a porous material.

The adsorbent 40a may adsorb a gas not to be detected contained in the sample gas. Examples of the adsorbent 40a that adsorbs a gas not to be detected include silica gel and zeolite.

The adsorbent 40b may adsorb a gas to be detected contained in the sample gas. Examples of the adsorbent 40b that adsorbs a gas to be detected include activated carbon and molecular sieve. However, the combination of them may be appropriately changed according to the polarity of gas molecules to be adsorbed.

In the storage tank 40, the adsorbent 40a may be disposed in locations separated by walls 40c. Separation of the locations in which the adsorbent 40a is positioned can lengthen the flow path of the gas in the storage tank 40. The flow path of the gas in the storage tank 40 is lengthened, which can lengthen the time during which the gas and the adsorbent 40a are in contact with each other. Likewise, in the storage tank 40, the adsorbent 40b may be separated by the walls 40c and disposed. Separation of the locations in which the adsorbent 40b is positioned can lengthen the time during which the gas and the adsorbent 40b are in contact with each other in the storage tank 40.

The adsorbent 40a may be provided on the side of the storage tank 40 where the storage tank 40 is connected to the suction hole 20. The adsorbent 40b may be provided on the side of the storage tank 40 where the storage tank 40 is connected to the flow path 23-1.

The storage tank 40 may be formed by a tank or the like having a rectangular parallelepiped shape, a cylindrical shape, a bag shape, or a shape such that it fits in a gap between various components housed inside the housing 10. The storage tank 40 may be provided with a heater for heating at least one of an inner wall of the storage tank 40 and the adsorbent 40a.

The entire storage tank 40 may be separated by the walls 40c. Separation of the entire storage tank 40 allows the flow path of the gas to have a small cross-sectional area relative to the volume of the flow path of the gas in the storage tank 40. The flow path of the gas has a small cross-sectional area relative to the volume of the flow path of the gas, which can reduce the contact area between the gas flowing into the storage tank 40 from the valve 20B and the sample gas stored in the storage tank 40 when the sample gas is pushed out into the chamber 30 from the storage tank 40. The contact area between the gas flowing into the storage tank 40 from the valve 20B and the sample gas stored in the storage tank 40 is reduced, which makes it less likely that the gas flowing into the storage tank 40 from the valve 20B is mixed with the sample gas in the storage tank 40.

As illustrated in FIG. 2, the storage tank 41 is connected to a connection port of the valve 21B. A portion of the storage tank 41 that is connected to the connection port of the valve 21B is also referred to as an “inlet portion”. The storage tank 41 is connected to the flow path 24-1. A portion of the storage tank 41 that is connected to the flow path 24-1 is also referred to as an “outlet portion”.

The storage tank 41 is capable of storing the purge gas. The purge gas stored in the storage tank 41 is supplied to the chamber 30 via the flow paths 24-1 and 24-2 and the supply unit 51. The residual gas or the like in the storage tank 41 can be discharged to the outside from the discharge path 22 via the flow path 24-1, the valve 26, the flow paths 27-2 and 27-3, and the supply unit 52.

An adsorbent 41a and an adsorbent 41b may be placed in the storage tank 41. Each of the adsorbent 41a and the adsorbent 41a may contain any material corresponding to the use. Each of the adsorbent 41a and the adsorbent 41b may contain, for example, at least any one of activated carbon, silica gel, zeolite, and molecular sieve. The adsorbent 41a and the adsorbent 41b may be of a plurality of types or may contain a porous material.

The adsorbent 41a may adsorb a gas not to be detected contained in the purge gas. When the air in the toilet room 100 is a purge gas, the purge gas may contain a gas not to be detected. Since the adsorbent 41a adsorbs a gas not to be detected contained in the purge gas, the purge gas in the storage tank 41 can be purified. Examples of the adsorbent 41a that adsorbs a gas not to be detected include silica gel and zeolite. The adsorbent 41b may adsorb a gas to be detected contained in the purge gas. When the air in the toilet room 100 is a purge gas, the purge gas may contain a gas to be detected. Since the adsorbent 41a adsorbs a gas to be detected contained in the purge gas, the purge gas in the storage tank 41 can be purified. Examples of the adsorbent 41b that adsorbs a gas to be detected include activated carbon and molecular sieve. However, the combination of them may be appropriately changed according to the polarity of gas molecules to be adsorbed.

In the storage tank 41, the adsorbent 41a may be separated by walls 41c and disposed. Separation of the adsorbent 41a can lengthen the flow path of the gas in the storage tank 41. The flow path of the gas in the storage tank 41 is lengthened, which can lengthen the time during which the gas and the adsorbent 41a are in contact with each other. Likewise, in the storage tank 41, the adsorbent 41b may be separated by the walls 41c and disposed. Separation of the adsorbent 41b can lengthen the time during which the gas and the adsorbent 41b are in contact with each other in the storage tank 41.

The adsorbent 41a may be provided on the side of the storage tank 41 where the storage tank 41 is connected to the suction hole 21. The adsorbent 41b may be provided on the side of the storage tank 41 where the storage tank 41 is connected to the flow path 24-1.

The storage tank 41 may be formed by a tank or the like having a rectangular parallelepiped shape, a cylindrical shape, a bag shape, or a shape such that it fits in a gap between various components housed inside the housing 10. The storage tank 41 may be provided with a heater for heating at least one of an inner wall of the storage tank 41, the adsorbent 41a, and the adsorbent 41b.

The entire storage tank 41 may be separated by the walls 41c. Separation of the entire storage tank 41 allows the flow path of the gas to have a small cross-sectional area relative to the volume of the flow path of the gas in the storage tank 41. The flow path of the gas has a small cross-sectional area relative to the volume of the flow path of the gas, which can reduce the contact area between the gas flowing into the storage tank 41 from the valve 21B and the purge gas stored in the storage tank 41 when the purge gas is pushed out into the chamber 30 from the storage tank 41. The contact area between the gas flowing into the storage tank 41 from the valve 21B and the purge gas stored in the storage tank 41 is reduced, which makes it less likely that the gas flowing into the storage tank 41 from the valve 21B is mixed with the purge gas in the storage tank 41. With this configuration, for example, if a gas near the suction hole 21 is contaminated, the contaminated gas is less likely to be mixed with the purge gas in the storage tank 41.

The supply unit 50 as illustrated in FIG. 2 is attached to the flow path 23-2. The supply unit 50 is capable of supplying the sample gas stored in the storage tank 40 to the chamber 30 when the valve 25 connects the flow path 23-1 and the flow path 23-2 to each other. For example, the supply unit 50 supplies the sample gas stored in the storage tank 40 to the chamber 30 at a predetermined timing under the control of the control unit 64. The arrow illustrated in the supply unit 50 indicates the direction in which the supply unit 50 sends the sample gas. The supply unit 50 may be constituted by a piezoelectric pump, a motor pump, or the like.

The supply unit 51 as illustrated in FIG. 2 is attached to the flow path 24-2. The supply unit 51 is capable of supplying the purge gas stored in the storage tank 41 to the chamber 30 when the valve 26 connects the flow path 24-1 and the flow path 24-2 to each other. For example, the supply unit 51 supplies the purge gas stored in the storage tank 41 to the chamber 30 at a predetermined timing under the control of the control unit 64. The arrow illustrated in the supply unit 51 indicates the direction in which the supply unit 51 sends the purge gas. The supply unit 51 may be constituted by a piezoelectric pump, a motor pump, or the like.

The supply unit 52 as illustrated in FIG. 2 is attached to the flow path 27-3. The supply unit 52 is capable of supplying the residual gas or the like in the storage tank 40 to the discharge path 22 when the valve 25 connects the flow path 23-1 and the flow path 27-1 to each other. Further, the supply unit 52 is capable of supplying the residual gas or the like in the storage tank 41 to the discharge path 22 when the valve 26 connects the flow path 24-1 and the flow path 27-2 to each other. The supply unit 52 supplies the residual gas or the like in at least any one of the storage tank 40 and the storage tank 41 to the discharge path 22 under the control of the control unit 64. The arrow illustrated in the supply unit 52 indicates the direction in which the residual gas or the like is sent to the discharge path 22. The supply unit 52 may be constituted by a piezoelectric pump, a motor pump, or the like.

The supply unit 52 is capable of supplying the sample gas from the suction hole 20 to the storage tank 40 when the valve 20B connects the suction hole 20 and the storage tank 40 to each other and the valve 25 connects the flow path 23-1 and the flow path 27-1 to each other. Further, the supply unit 52 is capable of supplying the purge gas from the suction hole 21 to the storage tank 41 when the valve 21B connects the suction hole 21 and the storage tank 41 to each other and the valve 26 connects the flow path 24-1 and the flow path 27-2 to each other.

The circuit board 60 as illustrated in FIG. 3 has mounted therein wiring through which an electrical signal propagates, the storage unit 61, the communication unit 62, the control unit 64, and the like.

The storage unit 61 as illustrated in FIG. 3 is constituted by, for example, a semiconductor memory, a magnetic memory, or the like. The storage unit 61 stores various kinds of information, a program for operating the gas detection system 1, and the like. The storage unit 61 may function as a work memory.

The communication unit 62 as illustrated in FIG. 3 is capable of communicating with the electronic device 3 and the ventilation fan 4 as illustrated in FIG. 1. The communication method used when the communication unit 62 communicates with the electronic device 3 and the ventilation fan 4 may be a short-range wireless communication standard, a wireless communication standard for connecting to a mobile phone network, or a wired communication standard. The short-range wireless communication standard may include, for example, WiFi (registered trademark), Bluetooth (registered trademark), infrared, NFC (Near Field Communication), and the like. The wireless communication standard for connecting to a mobile phone network may include, for example, LTE (Long Term Evolution) or a fourth generation or higher mobile communication system, and the like. Alternatively, the communication method used when the communication unit 62 communicates with the electronic device 3 and an external server may be, for example, a communication standard such as LPWA (Low Power Wide Area) or LPWAN (Low Power Wide Area Network).

The sensor unit 63 as illustrated in FIG. 3 may include at least any one of an image camera, a personal identification switch, an infrared sensor, a pressure sensor, a cleanliness sensor, and the like. The sensor unit 63 outputs a detection result to the control unit 64.

For example, when the sensor unit 63 includes an infrared sensor, the sensor unit 63 detects reflected light from an object irradiated with infrared radiation from the infrared sensor, thereby being able to detect that the subject has entered the toilet room 100. The sensor unit 63 outputs, as a detection result, a signal indicating that the subject has entered the toilet room 100 to the control unit 64.

For example, when the sensor unit 63 includes an infrared sensor, the sensor unit 63 detects reflected light from an object irradiated with infrared radiation from the infrared sensor, thereby being able to detect that the subject has exited the toilet room 100. The sensor unit 63 outputs, as a detection result, a signal indicating that the subject has exited the toilet room 100 to the control unit 64.

For example, when the sensor unit 63 includes a pressure sensor, the sensor unit 63 detects a pressure applied to the toilet seat 2B as illustrated in FIG. 1, thereby being able to detect that the subject has sat on the toilet seat 2B. The sensor unit 63 outputs, as a detection result, a signal indicating that the subject has sat on the toilet seat 2B to the control unit 64.

For example, when the sensor unit 63 includes a pressure sensor, the sensor unit 63 detects a reduction in the pressure applied to the toilet seat 2B as illustrated in FIG. 1, thereby being able to detect that the subject has risen from the toilet seat 2B. The sensor unit 63 outputs, as a detection result, a signal indicating that the subject has risen from the toilet seat 2B to the control unit 64.

For example, when the sensor unit 63 includes an image camera, a personal identification switch, and the like, the sensor unit 63 collects data, such as a face image, the sitting height, and the weight. The sensor unit 63 identifies and detects a person from the collected data. The sensor unit 63 outputs, as a detection result, a signal indicating the identified person to the control unit 64.

For example, when the sensor unit 63 includes a personal identification switch and the like, the sensor unit 63 identifies (detects) a person in response to an operation of the personal identification switch. In this case, personal information may be registered (stored) in the storage unit 61 in advance. The sensor unit 63 outputs, as a detection result, a signal indicating the identified person to the control unit 64.

For example, when the sensor unit 63 includes a cleanliness sensor, the sensor unit 63 detects the cleanliness of the air in the toilet room 100. The sensor unit 63 may detect the cleanliness of air outside the toilet bowl 2A as the air in the toilet room 100. The sensor unit 63 may have a configuration similar to that of the sensor unit 31 as illustrated in FIG. 2. In the present disclosure, the phrase “cleanliness of a gas is high” means that the concentration of the specific gas to be detected is low in the gas. The sensor unit 63 outputs, as a detection result, the cleanliness of the air in the toilet room 100 to the control unit 64.

The control unit 64 as illustrated in FIG. 3 includes one or more processors. The one or more processors may include at least any one of a general-purpose processor that reads a specific program to execute a specific function, and a dedicated processor dedicated to a specific process. The dedicated processor may include an application specific IC (ASIC; Application Specific Integrated Circuit). The one or more processors may include a programmable logic device (PLD; Programmable Logic Device). The PLD may include an FPGA (Field-Programmable Gate Array). The control unit 64 may include at least any one of an SoC (System-on-a-Chip) and an SiP (System-in-a-Package) with which the one or more processors cooperate.

The control unit 64 collects air in the toilet room 100 into a storage tank included in the gas detection system 1 as a sample gas or a purge gas. A period during which the control unit 64 collects the air in the toilet room 100 into the storage tank as a sample gas or a purge gas is also referred to as a “collection period”.

When the gas detection system 1 includes the storage tank 41 as a storage tank, the control unit 64 collects the air in the toilet room 100 into the storage tank 41 as a purge gas. In this embodiment, the control unit 64 collects a gas outside the toilet bowl 2A of the toilet 2 in the toilet room 100 into the storage tank 41 as a purge gas. For example, when the air blower 21A includes a fan, the control unit 64 causes the air blower 21A to rotate the fan of the air blower 21A to draw the purge gas into around the suction hole 21. The control unit 64 causes the valve 26 to connect the flow path 24-1 and the flow path 24-2 to each other and controls the supply unit 51 so that the purge gas drawn into around the suction hole 21 is sucked in through the suction hole 21. The control unit 64 causes the purge gas to be sucked in through the suction hole 21 and collected into the storage tank 41.

During a first period, the control unit 64 collects the air in the toilet room 100 into the storage tank 41 as a purge gas. In other words, the first period is a period during which the air in the toilet room 100 is collected into the storage tank 41 as a purge gas. The length of the first period may be appropriately set in consideration of the volumetric capacity of the storage tank 41 and the like. A setting example of the first period will be described below.

When the gas detection system 1 includes the storage tank 40 as a storage tank, the control unit 64 collects a sample gas into the storage tank 40. In this embodiment, the control unit 64 collects a gas inside the toilet bowl 2A of the toilet 2 in the toilet room 100 into the storage tank 40 as the sample gas. For example, when the air blower 20A includes a fan, the control unit 64 causes the air blower 20A to rotate the fan of the air blower 20A to draw the sample gas into around the suction hole 20. The control unit 64 causes the valve 25 to connect the flow path 23-1 and the flow path 23-2 to each other and controls the supply unit 50 so that the sample gas drawn into the suction hole 20 is sucked in through the suction hole 20. The control unit 64 causes the sample gas to be sucked in through the suction hole 20 and collected into the storage tank 40.

During a second period, the control unit 64 collects the air in the toilet room 100 into the storage tank 40 as a sample gas. In other words, the second period is a period during which the air in the toilet room 100 is collected into the storage tank 40 as a sample gas. The length of the second period may be appropriately set in consideration of the volumetric capacity of the storage tank 40 and the like. A setting example of the second period will be described below.

The control unit 64 supplies the sample gas stored in the storage tank 40 or the purge gas stored in the storage tank 41 to the sensor unit 31 in the chamber 30. A period during which the control unit 64 supplies the sample gas or the purge gas to the sensor unit 31 in the chamber 30 is also referred to as a “supply period”. In this embodiment, the control unit 64 controls the supply unit 50 and the supply unit 51 to alternately supply the sample gas stored in the storage tank 40 and the purge gas stored in the storage tank 41 to the chamber 30.

For example, when supplying the sample gas to the chamber 30, the control unit 64 causes the valve 25 to connect the flow path 23-1 and the flow path 23-2 to each other. The control unit 64 causes the valve 25 to connect the flow path 23-1 and the flow path 23-2 to each other and controls the supply unit 50 to supply the sample gas in the storage tank 40 to the chamber 30. When supplying the purge gas to the chamber 30, the control unit 64 causes the valve 26 to connect the flow path 24-1 and the flow path 24-2 to each other. The control unit 64 causes the valve 26 to connect the flow path 24-1 and the flow path 24-2 to each other and controls the supply unit 51 to supply the purge gas in the storage tank 41 to the chamber 30. However, the control process of the control unit 64 to supply the sample gas and the purge gas to the chamber 30 is not limited to this. For example, the control unit 64 may cause the valve 20B to connect the storage tank 40 and the flow path 28 to each other and cause the valve 26 to connect the flow path 24-1 and the flow path 28 to each other to supply the purge gas in the storage tank 41 to the storage tank 40 from the valve 20B side. The control unit 64 may supply the purge gas to the storage tank 40 such that the sample gas in the storage tank 40 is pushed out toward the flow path 23-1 by the purge gas to supply the sample gas in the storage tank 40 to the chamber 30.

The control unit 64 alternately supplies the purge gas and the sample gas to the chamber 30 to acquire a voltage waveform from the sensor unit 31. The control unit 64 detects the type and concentration of a gas contained in the sample gas on the basis of the voltage waveform. For example, the control unit 64 detects the type and concentration of a gas contained in the sample gas by, for example, machine learning for the voltage waveform acquired from the sensor unit 31. The control unit 64 may transmit the detected type and concentration of the gas to the electronic device 3 via the communication unit 62 as a detection result.

Here, the control unit 64 sets a period during which the air in the toilet room 100 is collected into a storage tank as a sample gas or a purge gas and a supply period during which the sample gas or the purge gas is supplied to the sensor unit 31 in the chamber 30 such that the period and the supply period fall in different time slots. Setting examples of the periods will be described hereinafter.

### Period Setting Example 1

The control unit 64 may set a first period during which the air in the toilet room 100 is collected into the storage tank 41 as a purge gas and a supply period during which the purge gas stored in the storage tank 41 is supplied to the sensor unit 31 in the chamber 30 such that the first period and the supply period fall in different time slots.

In one example, the control unit 64 may periodically supply the air in the toilet room 100 to the sensor unit 31 in the chamber 30. A regular period during which the air in the toilet room 100 is supplied to the sensor unit 31 may be appropriately set in consideration of the frequency of use of the toilet 2. The control unit 64 may control the supply unit 51 to periodically supply the air in the toilet room 100 to the chamber 30 through the suction hole 21 and the storage tank 41. Further, the control unit 64 may set the first period during which the purge gas is collected into the storage tank 41 on the basis of a detection result periodically output from the sensor unit 31. For example, the control unit 64 may set, as a point in time at which the first period starts, a point in time at which the cleanliness of the air in the toilet room 100 is determined to exceed a predetermined value, on the basis of the detection result of the sensor unit 31. The predetermined value may be appropriately set in consideration of the cleanliness of a gas that can serve as a purge gas. With this configuration, air in the toilet room 100 having high cleanliness can be collected into the storage tank 41 as a purge gas. In addition, the control unit 64 may set the supply period described above to fall in a time slot later than the set first period to set the one period and the supply period to fall in different time slots. With this configuration, the air in the toilet room 100 can be collected into the storage tank 41 as a purge gas before the supply period. Collecting the purge gas before the supply period eliminates, in this embodiment, the need to prepare the purge gas by using a cylinder or the like. In this embodiment, there is no need to use a cylinder or the like, which can reduce the probability of an increase in the size of the device due to installation of the cylinder or the like and the probability of an increase in cost due to preparation of the cylinder or the like.

In another example, the control unit 64 may set, as a point in time at which the first period starts, a point in time at which a predetermined time elapses after the subject exits the toilet room 100, on the basis of a detection result of the sensor unit 63. The length of the predetermined time may be appropriately set by taking into account a time period until the cleanliness of the air in the toilet room 100 exceeds a predetermined value after the subject exits the toilet room 100. The predetermined value may be appropriately set in consideration of the cleanliness of a gas that can serve as a purge gas in the gas detection system 1. In addition, the control unit 64 may set the supply period described above to fall in a time slot later than the set first period to set the one period and the supply period to fall in different time slots. With this configuration, as described above, there is no need to use a cylinder or the like, which can reduce the probability of an increase in the size of the device due to installation of the cylinder or the like and the probability of an increase in cost due to preparation of the cylinder or the like.

In still another example, the control unit 64 may set, as a point in time at which the first period starts, a point in time at which the cleanliness of the air in the toilet room 100 is determined to exceed a predetermined value on the basis of the detection result of the sensor unit 63. The predetermined value may be appropriately set in consideration of the cleanliness of a gas that can serve as a purge gas. In addition, the control unit 64 may set the supply period described above to fall in a time slot later than the set first period to set the first period and the supply period to fall in different time slots. With this configuration, as described above, there is no need to use a cylinder or the like, which can reduce the probability of an increase in the size of the device due to installation of the cylinder or the like and the probability of an increase in cost due to preparation of the cylinder or the like.

### Period Setting Example 2

The control unit 64 may set a second period during which the air in the toilet room 100 is collected into the storage tank 40 as a sample gas and a supply period during which the sample gas stored in the storage tank 40 is supplied to the sensor unit 31 in the chamber 30 such that the second period and the supply period fall in different time slots.

In one example, the control unit 64 may periodically supply the air in the toilet room 100 to the sensor unit 31 in the chamber 30. A regular period during which the air in the toilet room 100 is supplied to the sensor unit 31 may be appropriately set in consideration of the frequency of use of the toilet 2. The control unit 64 may control the supply unit 51 to periodically supply the air in the toilet room 100 to the chamber 30 through the suction hole 21 and the storage tank 41. Further, the control unit 64 may set the second period during which the sample gas is collected into the storage tank 40 on the basis of a detection result periodically output from the sensor unit 31. For example, the control unit 64 may set, as the second period, a period during which a gas to be detected contained in the air in the toilet room 100 exceeds a predetermined amount, on the basis of the detection result of the sensor unit 31. The predetermined amount may be appropriately set in consideration of the volumetric capacity of the toilet room 100 and the like. With this configuration, the sample gas can be collected into the storage tank 40 while, for example, the subject is using the toilet 2. In addition, the control unit 64 may set the supply period during which the sample gas stored in the storage tank 40 is supplied to the sensor unit 31 on the basis of a detection result periodically output from the sensor unit 31. For example, the control unit 64 may set, as the supply period, a period during which the gas to be detected contained in the air in the toilet room 100 falls below the predetermined amount, on the basis of the detection result of the sensor unit 31. With this configuration, for example, when the subject is not using the toilet 2, the sample gas in the storage tank 40 is supplied to the sensor unit 31, and detection of the concentration and type of the gas contained in the sample gas can be performed. Here, for example, if the subject uses the toilet 2 in a short time, the gas detection system 1 may fail to perform collection of the sample gas into the storage tank 40 and detection of the type and concentration of the gas contained in the sample gas while the subject is using the toilet 2. Even in this case, the control described above allows the gas detection system 1 to collect the sample gas into the storage tank 40 while the subject is using the toilet 2. Further, after the subject uses the toilet 2, the gas detection system 1 can use the sample gas stored in the storage tank 40 to detect the type and concentration of the gas contained in the sample gas. Accordingly, for example, even if the subject uses the toilet 2 in a short time, the control described above allows the gas detection system 1 to collect the sample gas and detect the type and concentration of the gas contained in the sample gas.

In still another example, the control unit 64 may set the second period on the basis of a detection result of the sensor unit 63. For example, the control unit 64 may set, as a point in time at which the second period start, a point in time at which a certain time elapses after it is detected that the subject has sat on the toilet seat 2B, on the basis of the detection result of the sensor unit 63. The length of the certain time may be appropriately set in consideration of the time period from when the subject sits on the toilet seat 2B to when the subject starts defecating. With this configuration, the second period can be a period during which the subject is defecating using the toilet 2. Further, the control unit 64 may set the supply period described above to fall in a time slot later than the set second period to set the second period and the supply period to fall in different time slots.

### Period Setting Example 3

The control unit 64 may set the first period and the second period to fall in different time slots. The second period is likely to be a period during which the subject is defecating using the toilet 2. That is, during the second period, the sample gas generated from feces in the toilet bowl 2A may be likely to leak also to outside the toilet bowl 2A. In this embodiment, setting the first period and the second period to fall in different time slots allows a purge gas having higher cleanliness to be collected into the storage tank 41.

In one example, the control unit 64 may set the first period in accordance with exit of the subject from the toilet room 100. As described above, the second period can be a period during which the subject is defecating using the toilet 2. Accordingly, setting the first period in accordance with exit of the subject from the toilet room 100 allows the first period to be set to fall in a time slot different from that of the second period. Specifically, the control unit 64 may set, as a point in time at which the first period starts, a point in time at which a predetermined time elapses after the subject exits the toilet room 100, on the basis of the detection result of the sensor unit 63. The length of the predetermined time may be appropriately set in consideration of a time period until the cleanliness of the air in the toilet room 100 exceeds a predetermined value after the subject exits the toilet room 100. The predetermined value may be appropriately set in consideration of the cleanliness of a gas that can serve as a purge gas in the gas detection system 1.

In another example, the control unit 64 may set, as a point in time at which the first period starts, a point in time at which the cleanliness of the air in the toilet room 100 is determined to exceed a predetermined value on the basis of the detection result of the sensor unit 63. The predetermined value may be appropriately set in consideration of the cleanliness of a gas that can serve as a purge gas. As described above, the second period is likely to be a period during which the subject is defecating using the toilet 2. When the cleanliness of the air in the toilet room 100 exceeds the predetermined value, the subject is likely not to be using the toilet 2. The point in time at which the control unit 64 determines that the cleanliness of the air in the toilet room 100 exceeds the predetermined value is set as a point in time at which the first period starts. Thus, the first period can be set to fall in a time slot different from that of the second period. The control unit 64 may set, as a point in time at which the first period starts, a point in time at which the cleanliness of the air in the toilet room 100 is determined to exceed a predetermined value on the basis of the detection result of the sensor unit 31 instead of the sensor unit 63. In this case, the control unit 64 may control the supply unit 51 to supply the air in the toilet room 100 to the chamber 30 through the suction hole 21 and the storage tank 41.

In still another example, the control unit 64 may set the first period in accordance with the operating state of the ventilation fan 4 in the toilet room 100. This example may be adopted in a case where the ventilation fan 4 is set to enter the driven state after the subject exits the toilet room 100. Setting the first period in accordance with the operating state of the ventilation fan 4 allows the first period to be set to fall in a time slot different from that of the second period. The control unit 64 may set, as a point in time at which the first period starts, a point in time at which the ventilation fan 4 is determined to be in the operating state, through communication with the ventilation fan 4 via the communication unit 62. The operation of the ventilation fan 4 can increase the cleanliness of the air in the toilet room 100. Accordingly, setting the first period in accordance with the operating state of the ventilation fan 4 allows the purge gas having higher cleanliness to be collected.

[Example Operation of Gas Detection System]

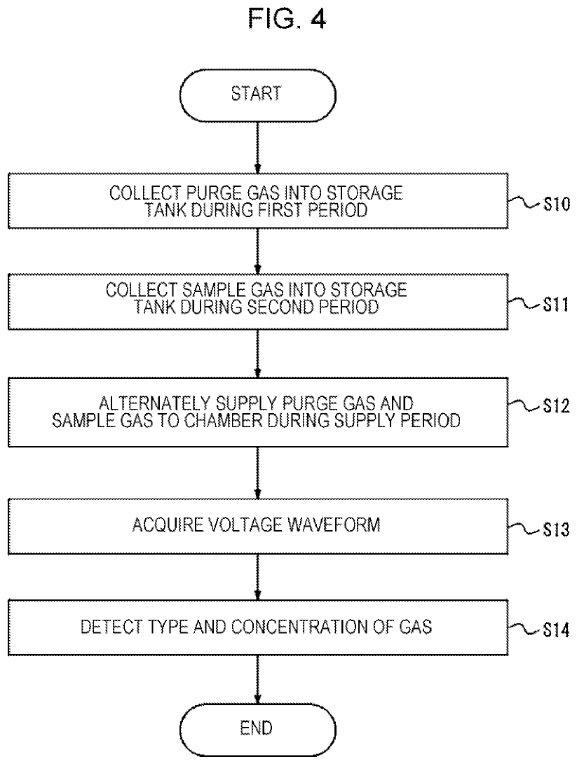

FIG. 4 is a flowchart illustrating the operation of the gas detection system 1 according to the first embodiment of the present disclosure.

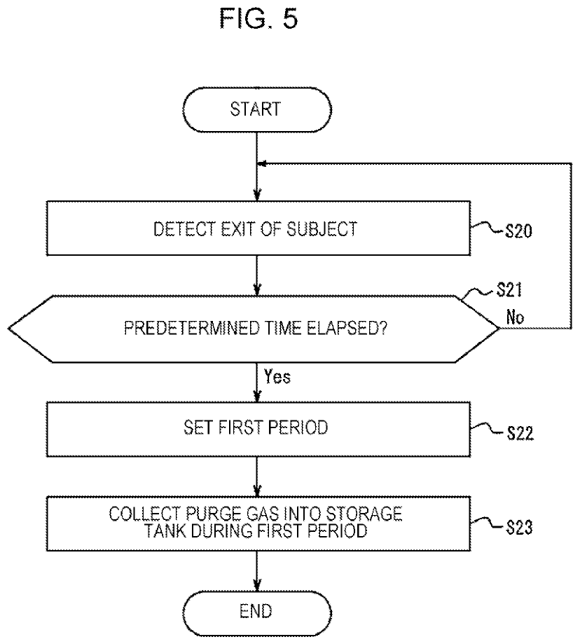

During the first period, the control unit 64 collects a purge gas into the storage tank 41 (step S10). An example of the details of the processing of step S10 will be described below with reference to FIGS. 5 to 7. During the second period, the control unit 64 collects a sample gas into the storage tank 40 (step S11).

The control unit 64 alternately supplies the sample gas stored in the storage tank 40 and the purge gas stored in the storage tank 41 to the chamber 30 during the supply period (step S12).

The control unit 64 alternately supplies the purge gas and the sample gas to the chamber 30 to acquire a voltage waveform from the sensor unit 31 (step S13).

The control unit 64 detects the type and concentration of a gas contained in the sample gas on the basis of the voltage waveform acquired from the sensor unit 31 in the processing of step S13 (step S14).

FIG. 5 is a flowchart illustrating an example operation of the gas detection system 1 according to the first embodiment of the present disclosure during collection of the purge gas. A process as illustrated in FIG. 5 corresponds to an example of the processing of step S10 as illustrated in FIG. 4.

The control unit 64 detects that the subject has exited the toilet room 100 on the basis of the detection result of the sensor unit 63 (step S20). The control unit 64 determines whether a predetermined time has elapsed since the subject exited the toilet room 100 (step S21).

If the control unit determines that the predetermined time has elapsed since the subject exited the toilet room 100 (step S21: Yes), the control unit 64 sets, as a point in time at which the first period starts, a point in time at which the predetermined time has elapsed since the subject exited the toilet room 100 (step S22). On the other hand, if the control unit 64 does not determine that the predetermined time has elapsed since the subject exited the toilet room 100 (step S21: No), the control unit 64 executes the processing of step S21 again.

In the processing of step S23, the control unit 64 collects the air in the toilet room 100 into the storage tank 41 as a purge gas during a first period.

When the control unit 64 detects that the subject has entered the toilet room 100 on the basis of the detection result of the sensor unit 63 before executing the processing of step S23, the control unit 64 may not execute the processing of step S23.

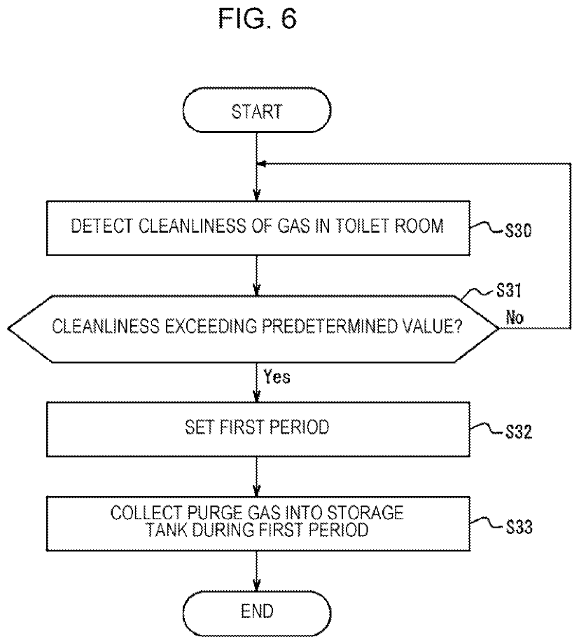

FIG. 6 is a flowchart illustrating another example operation of the gas detection system 1 according to the first embodiment of the present disclosure during collection of the purge gas. A process as illustrated in FIG. 6 corresponds to another example of the processing of step S10 as illustrated in FIG. 4.

The control unit 64 detects the cleanliness of the air in the toilet room 100 on the basis of the detection result of the sensor unit 63 (step S30). The control unit 64 determines whether the cleanliness of the air in the toilet room 100 exceeds a predetermined value (step S31). If the control unit 64 determines that the cleanliness of the air in the toilet room 100 exceeds the predetermined value (step S31: Yes), the control unit 64 sets, as a point in time at which the first period starts, a point in time at which the cleanliness of the air in the toilet room 100 is determined to exceed the predetermined value (step S32). On the other hand, if the control unit 64 does not determine that the cleanliness of the air in the toilet room 100 exceeds the predetermined value (step S31: No), the control unit 64 returns to the processing of step S30.

In the processing of step S33, the control unit 64 collects the air in the toilet room 100 into the storage tank 41 as a purge gas during the first period.

In the processing of step S30, the control unit 64 may detect the cleanliness of the air in the toilet room 100 on the basis of the detection result of the sensor unit 31. In this case, the control unit 64 may control the supply unit 51 to supply the air in the toilet room 100 to the chamber 30 through the suction hole 21 and the storage tank 41.

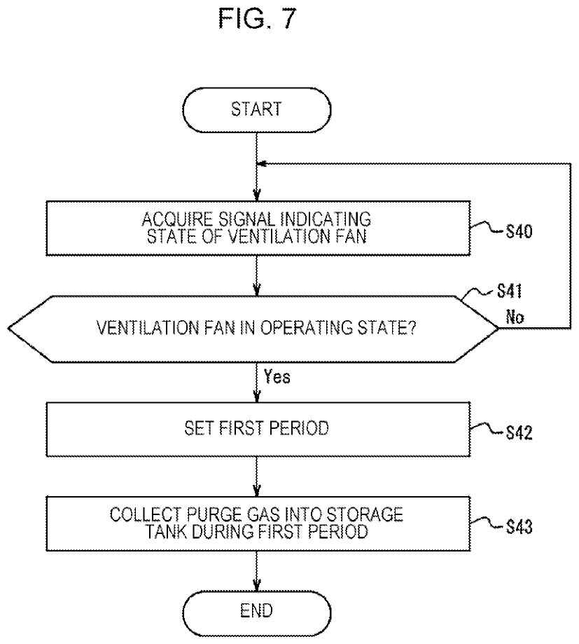

FIG. 7 is a flowchart illustrating still another example operation of the gas detection system 1 illustrated in FIG. 1 during collection of the purge gas. A process as illustrated in FIG. 7 corresponds to still another example of the processing of step S10 as illustrated in FIG. 4.

The control unit 64 acquires a signal indicating the state of the ventilation fan 4 from the ventilation fan 4 via the communication unit 62 (step S40). The control unit 64 determines whether the ventilation fan 4 is in the operating state on the basis of the acquired signal indicating the state of the ventilation fan 4 (step S41). If the control unit 64 determines that the ventilation fan 4 is in the operating state (step S41: Yes), the control unit 64 sets, as a point in time at which the first period starts, for example, a point in time at which the ventilation fan 4 is determined to be in the operating state (step S42). On the other hand, if the control unit 64 does not determine that the ventilation fan 4 is in the operating state (step S41: No), the control unit 64 returns to the processing of step S40.

In the processing of step S43, the control unit 64 collects the air in the toilet room 100 into the storage tank 41 as a purge gas during the first period.

As described above, in the first embodiment, the control unit 64 sets a collection period during which the air in the toilet room 100 is collected into a storage tank as a sample gas or a purge gas and a supply period during which the sample gas or the purge gas is supplied to the sensor unit 31 in the chamber 30 such that the collection period and the supply period fall in different time slots. With this configuration, for example, setting a first period during which the air in the toilet room 100 is collected into the storage tank 41 as a purge gas and the supply period to fall in different time slots eliminates the need to prepare the purge gas by using a cylinder or the like, as described above. Accordingly, the gas detection system 1 according to this embodiment can reduce the probability of an increase in the size of the device due to installation of the cylinder or the like and the probability of an increase in cost due to preparation of the cylinder or the like.

According to this embodiment, therefore, the improved gas detection system 1 can be provided.

### Second Embodiment

[Example Configuration of Gas Detection System]

A gas detection system according to a second embodiment can adopt a configuration similar to that of the gas detection system 1 as illustrated in FIGS. 1 to 3. A gas detection system 1 according to the second embodiment will be described hereinafter with reference to FIGS. 1 to 3.

In the second embodiment, the control unit 64 executes refresh processing on the sensor unit 31 twice. The refresh processing is processing for removing substances adhering to the sensor unit 31.

In the first refresh processing, the control unit 64 executes refresh processing on the sensor unit 31 by using, for example, the air in the toilet room 100, which is acquired at any timing. The control unit 64 may execute the first refresh processing at any timing. The control unit 64 controls the supply unit 51 to supply the air in the toilet room 100 to the chamber 30 through the suction hole 21 and the storage tank 41. The control unit 64 supplies the air in the toilet room 100 to the chamber 30 to supply the air in the toilet room 100 to the sensor unit 31. When the air in the toilet room 100 is supplied to the sensor unit 31, substances adhering to the sensor unit 31 can be removed to some extent.

Alternatively, in the first refresh processing, the control unit 64 may execute refresh processing on the sensor unit 31 by using a gas remaining in the storage tank 41. In this case, the control unit 64 controls the supply unit 51 to supply the gas remaining in the storage tank 41 to the chamber 30.

In the second refresh processing, the control unit 64 executes refresh processing on the sensor unit 31 by using the purge gas stored in the storage tank 41. For example, the control unit 64 may execute the second refresh processing immediately before the sensor unit 31 executes gas detection processing. The control unit 64 may perform the processing described above in the first embodiment to collect the purge gas into the storage tank 41. The cleanliness of the purge gas stored in the storage tank 41 is higher than the degree of cleaning of the air or the like in the toilet room 100, which is used in the first refresh processing. Refresh processing executed on the sensor unit 31 by using the purge gas having higher degree of cleaning allows further removal of substances adhering to the sensor unit 31.

[Example Operation of Gas Detection System]

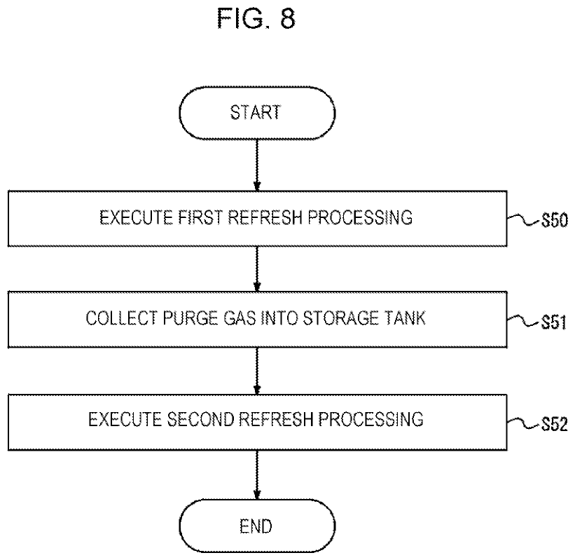

FIG. 8 is a flowchart illustrating the operation of the gas detection system 1 according to the second embodiment of the present disclosure during refresh. The control unit 64 may execute a process as illustrated in FIG. 8 after the gas detection processing, that is, after the process as illustrated in FIG. 4 is completed.

The control unit 64 executes the first refresh processing on the sensor unit 31 by using the air in the toilet room 100 (step S50).

The control unit 64 executes the process as illustrated in any one of FIGS. 5 to 7 to collect the air in the toilet room 100 into the storage tank 41 as a purge gas (step S51).

The control unit 64 executes refresh processing on the sensor unit 31 by using the purge gas stored in the storage tank 41 (step S52).

In the processing of step S50, the control unit 64 may execute the first refresh processing on the sensor unit 31 by using a gas remaining in the storage tank 41.

As described above, in the second embodiment, the control unit 64 executes the first refresh processing on the sensor unit 31 by using the air in the toilet room 100 and then executes the second refresh processing on the sensor unit 31 by using the purge gas stored in the storage tank 41. With this configuration, the gas detection system 1 according to this embodiment can execute refresh processing on the sensor unit 31 while saving the purge gas stored in the storage tank 41.

The drawings describing an embodiment according to the present disclosure are schematic ones. Dimensional ratios and the like in the drawings do not necessarily match the actual ones.

While embodiments according to the present disclosure have been described with reference to the drawings and examples, it should be noted that various modifications or changes can be easily made by a person skilled in the art on the basis of the present disclosure. Accordingly, it should be noted that these modifications or changes fall within the scope of the present disclosure. For example, the functions and the like included in each component or the like can be rearranged in any manner that is not logically contradictory, and a plurality of components or the like may be combined into one or divided.

For example, in the embodiments described above, the control unit 64 as illustrated in FIG. 3 has been described as being configured to control the supply unit 50 as illustrated in FIG. 2 to cause a sample gas to be sucked in through the suction hole 20 and collected into the storage tank 40. However, the process of the control unit 64 for collecting the sample gas into the storage tank 40 is not limited to this. For example, the control unit 64 may control the supply unit 52 to cause the sample gas to be sucked in through the suction hole 20 and collected into the storage tank 40. In this case, the control unit 64 causes the valve 20B to connect the suction hole 20 and the storage tank 40 to each other and causes the valve 25 to connect the flow path 23-1 and the flow path 27-1 to each other. The control unit 64 further controls the supply unit 52 to cause the sample gas to be sucked in through the suction hole 20 and collected into the storage tank 40.

For example, in the embodiments described above, the control unit 64 as illustrated in FIG. 3 has been described as being configured to control the supply unit 51 as illustrated in FIG. 2 to cause the purge gas to be sucked in through the suction hole 21 and collected into the storage tank 41. However, the process of the control unit 64 for collecting the purge gas into the storage tank 41 is not limited to this. For example, the control unit 64 may control the supply unit 52 to cause the purge gas to be sucked in through the suction hole 21 and collected into the storage tank 41. In this case, the control unit 64 causes the valve 21B to connect the suction hole 21 and the storage tank 41 to each other and causes the valve 26 to connect the flow path 24-1 and the flow path 27-2 to each other. The control unit 64 further controls the supply unit 52 to cause the purge gas to be sucked in through the suction hole 21 and collected into the storage tank 41.

For example, in the embodiments described above, the ventilation fan 4 has been described as being set to enter the driven state after the subject exits the toilet room 100. However, the timing at which the ventilation fan 4 enters the driven state is not limited to this. The ventilation fan 4 may enter the driven state at any timing regardless of whether the subject is present or absent in the toilet room 100. In this case, when the ventilation fan 4 is in the driven state, the control unit 64 may collect a gas outside the toilet bowl 2A of the toilet 2 in the toilet room 100 into the storage tank 41 as a purge gas. The control unit 64 may detect the time at which the ventilation fan 4 is in the operating state, through communication with the ventilation fan 4 via the communication unit 62. Alternatively, after a certain time elapses after it is detected that the subject has sat on the toilet seat 2B on the basis of the detection result of the sensor unit 63, the control unit 64 may collect a gas inside the toilet bowl 2A of the toilet 2 in the toilet room 100 into the storage tank 40 as a sample gas.

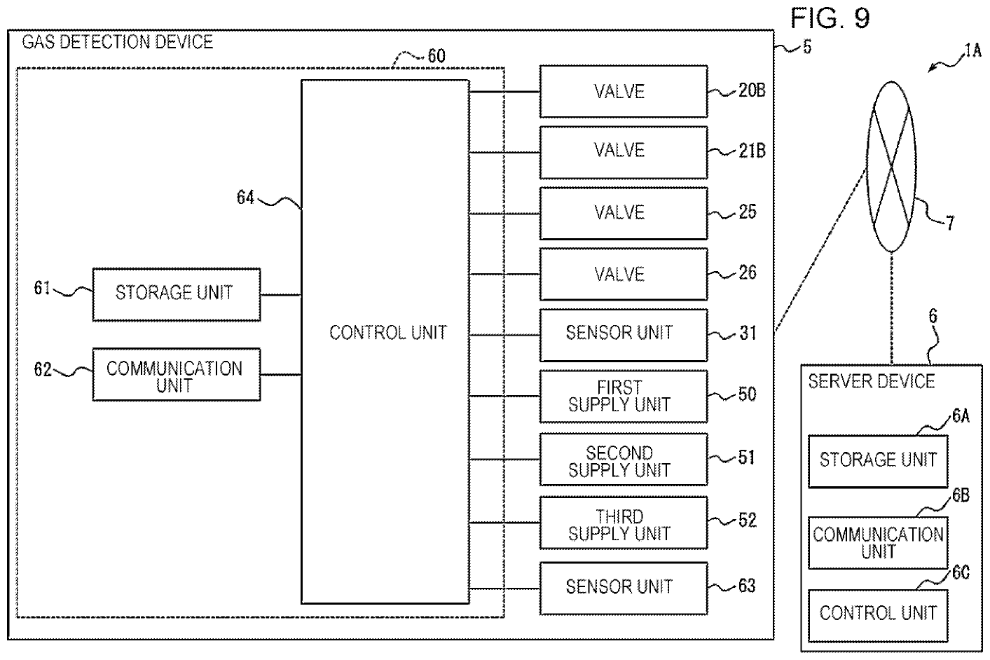

For example, in the embodiments described above, as illustrated in FIG. 3, the gas detection system 1 has been described as a single device. However, the gas detection system according to the present disclosure is not limited to the single device and may include a plurality of independent devices. The gas detection system according to the present disclosure may have, for example, a configuration as illustrated in FIG. 9.

As illustrated in FIG. 9, a gas detection system 1A includes a gas detection device 5 and a server device 6. The gas detection device 5 and the server device 6 are capable of communicating with each other via a network 7. A portion of the network 7 may be wired or wireless. The gas detection device 5 has a configuration similar to the configuration of the gas detection system 1 as illustrated in FIG. 2 and FIG. 3. The server device 6 includes a storage unit 6A, a communication unit 6B, and a control unit 6C. The control unit 6C is capable of executing the processes of the control unit 64 as illustrated in FIG. 3 described above. For example, the control unit 6C sets a collection period during which the air in the toilet room 100 is collected into a storage tank as a sample gas or a purge gas and a supply period during which the sample gas or the purge gas is supplied to the sensor unit 31 in the chamber 30 such that the collection period and the supply period fall in different time slots.

In the present disclosure, descriptions such as “first” and “second” are identifiers for distinguishing the respective configurations. The configurations distinguished by the descriptions such as “first” and “second” in the present disclosure may be interchangeably numbered. For example, a first storage tank and a second storage tank may exchange their identifiers “first” and “second”. The identifiers are exchanged simultaneously. Even after the identifiers are exchanged, the respective configurations are distinguishable. The identifiers may be deleted. Configurations without identifiers are distinguished using reference numerals. Only the description of identifiers such as “first” and “second” in the present disclosure should not be used for interpreting the order of the configurations or as a basis of the presence of identifiers with smaller numbers.

### REFERENCE SIGNS LIST

1, 1A gas detection system
2 toilet
2A toilet bowl
2B toilet seat
3 electronic device
3A display unit
4 ventilation fan
5 gas detection device
6 server device
6A storage unit
6B communication unit
6C control unit
7 network
10 housing
20, 21 suction hole
20A, 21A air blower
20B, 21B, 25, 26 valve
22 discharge path
23, 23-1, 23-2, 24, 24-1, 24-2, 27, 27-1, 27-2, 27-3, 27-4, 28, 29B flow path
30 chamber
31 sensor unit (first sensor unit)
40 storage tank (first storage tank)
40a, 40b adsorbent
40c wall
41 storage tank (second storage tank)
41a, 41b adsorbent
41c wall
50, 51, 52 supply unit
60 circuit board
61 storage unit
62 communication unit
63 sensor unit (second sensor unit)
64 control unit
100 toilet room (predetermined space)

1. A gas detection system comprising:
a first sensor unit that outputs a voltage corresponding to a concentration of a specific gas;
a storage tank capable of storing a sample gas or a purge gas to be supplied to the first sensor unit; and
a control unit that detects a type and a concentration of a gas contained in the sample gas based on a detection result of the first sensor unit, wherein
the control unit sets a collection period during which a gas in a predetermined space is collected into the storage tank as the sample gas or the purge gas and a supply period during which the sample gas or the purge gas is supplied to the first sensor unit such that the collection period and the supply period fall in different time slots.

2. The gas detection system according to claim 1, wherein
the storage tank includes a first storage tank capable of storing the purge gas, and
the control unit sets a first period and the supply period to fall in different time slots, the first period being the collection period during which the gas in the predetermined space is collected into the first storage tank as the purge gas.

3. The gas detection system according to claim 2, wherein
the storage tank includes a second storage tank capable of storing the sample gas, and
the control unit sets a second period and the supply period to fall in different time slots, the second period being the collection period during which the gas in the predetermined space is collected into the second storage tank as the sample gas.

4. The gas detection system according to claim 3, wherein
the control unit periodically supplies the gas in the predetermined space to the first sensor unit, and sets the second period based on a detection result periodically output from the first sensor unit.

5. The gas detection system according to claim 2, wherein
the control unit periodically supplies the gas in the predetermined space to the first sensor unit, and sets, as a point in time at which the first period starts, a point in time at which cleanliness of the gas in the predetermined space is determined to exceed a predetermined value based on a detection result periodically output from the first sensor unit.

6. The gas detection system according to claim 2, further comprising
a second sensor unit that detects entry or exit of a user into or from the predetermined space, wherein
the control unit sets, as a point in time at which the first period starts, a point in time at which a predetermined time elapses after it is detected that a user has exited the predetermined space based on a detection result of the second sensor unit.

7. The gas detection system according to claim 2, further comprising
a second sensor unit that detects cleanliness of the gas in the predetermined space, wherein
the control unit sets, as a point in time at which the first period starts, a point in time at which the cleanliness of the gas in the predetermined space is determined to exceed a predetermined value based on a detection result of the second sensor unit.

8. The gas detection system according to claim 2, wherein
the predetermined space is provided with a ventilation fan capable of exchanging the gas in the predetermined space with a gas outside the predetermined space, and
the control unit sets a point in time at which the first period starts in accordance with a driven state of the ventilation fan.

9. The gas detection system according to claim 2, wherein
the first storage tank has an adsorbent that adsorbs a gas not to be detected.

10. The gas detection system according to claim 2, wherein
the predetermined space is a toilet room, and
the control unit collects a gas outside a toilet bowl of a toilet in the toilet room into the first storage tank as the purge gas.

11. The gas detection system according to claim 3, wherein
the predetermined space is a toilet room, and
the control unit collects a gas inside a toilet bowl of a toilet in the toilet room into the second storage tank as the sample gas.

12. The gas detection system according to claim 2, wherein
the control unit
executes first refresh processing on the first sensor unit by using the gas in the predetermined space, and
executes second refresh processing on the first sensor unit by using the purge gas stored in the first storage tank.

13. The gas detection system according to claim 2, wherein
the control unit
executes first refresh processing on the first sensor unit by using a gas remaining in the first storage tank, and
executes second refresh processing on the first sensor unit by using the purge gas stored in the first storage tank.

14. A gas detection system comprising:
a first sensor unit that outputs a voltage corresponding to a concentration of a specific gas;
a first storage tank capable of storing a purge gas to be supplied to the first sensor unit; and
a control unit that collects a gas in a predetermined space into the first storage tank as the purge gas when a ventilation fan installed in the predetermined space is in a driven state, wherein
the ventilation fan is capable of exchanging the gas in the predetermined space with a gas outside the predetermined space.

15. A gas detection system comprising:
a first sensor unit that outputs a voltage corresponding to a concentration of a specific gas;
a first storage tank capable of storing a purge gas to be supplied to the first sensor unit; and
a control unit that detects a type and a concentration of a gas contained in a sample gas based on a detection result of the first sensor unit, wherein
the control unit
executes first refresh processing on the first sensor unit by using a gas remaining in the first storage tank or by using a gas in a predetermined space, and
executes second refresh processing on the first sensor unit by using the purge gas stored in the first storage tank.
